(19) United States
(12) Patent Application Publication
(10) Pub. No.: US 2010/0106008 A1
(43) Pub. Date: Apr. 29, 2010

(54) MAGNETIC RESONANCE IMAGING SYSTEM AND METHOD

(75) Inventor: Paul Royston Harvey, Eindhoven (NL)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N.V., EINDHOVEN (NL)

(21) Appl. No.: 12/531,360

(22) PCT Filed: Mar. 13, 2008
(86) PCT No.: PCT/IB08/50923

§ 371 Date: Sep. 15, 2009

(30) Foreign Application Priority Data
Mar. 20, 2007 (EP) 07104444.0
Mar. 23, 2007 (EP) 07104796.3

(51) Int. Cl. A61B 5/055

(52) U.S. Cl. 600/422

(57) ABSTRACT

It is an object of the present invention to provide a MRI technique, in which the influence of physiological factors such as respiration and cardiac pulsation on MRI results is reduced or removed. The object of the present invention is achieved by a magnetic resonance imaging system (1), comprising a first RF coil (2) adapted for acquiring magnetic resonance imaging data of a patient s body; a number of measuring elements (5, 5′, 5″, 29, 31), which are sensitive to a load changing of the first RF coil (2), said measuring elements (5, 5′, 5″, 29, 31) being adapted for acquiring data related to motion of said patient s body; and a processing unit (9) adapted for employing said motion data to correct for patient motion in magnetic resonance imaging.

### FIELD OF THE INVENTION

The present invention relates generally to non-invasive imaging applications, in particular to magnetic resonance imaging (MRI). More particularly, the present invention relates to an imaging technique employing radio frequency (RF) coils to measure properties of a patient's body being imaged.

### BACKGROUND OF THE INVENTION

MRI measures various magnetic properties of target material in a magnetic field. MRI includes aligning the spin of nuclei of material being imaged in a generally homogeneous magnetic field and perturbing the magnetic field with periodic RF pulses in order to measure the nuclear magnetic resonance (NMR) phenomenon of the material being imaged. To invoke the NMR phenomenon, one or more resonant coils are provided that generate the RF pulses at a resonant frequency that matches a Larmor frequency (i.e. the rate at which a nucleus precesses about an axis) of certain tissue in order to excite the nuclei such that they precess about an axis in the direction of the applied RF pulse. When the RF pulse subsides, the nuclei realign with the magnetic field, releasing energy that can be measured.

When a resonant coil is placed in proximity of a load, for example, a patient or other object to be imaged, various properties of the resonant coil may be affected. In MRI, this loading effect tends to negatively impact the operation of the device by altering the resonant frequency of the coil and causing other generally undesirable changes in the coil properties. This loading effect depends in part on the dielectric properties of the load. Changes in resonant frequency of the coil may reduce the device's ability to excite the nuclei of the material being imaged (e.g. by creating a mismatch between the coil's resonant frequency and the Larmor frequency of the target material) and negatively impact the quality of the resulting images. The effects of coil loading complicate MRI to the extent that resonant coils are often tuned or adjusted to compensate for the generally undesirable loading effect caused by the body being imaged.

In order to tune or adjust the resonant coil it is known from the prior art to employ an additional small RF coil inside of the resonant coil. The small RF coil measures a voltage, which depends on the local RF field of the resonant coil, which is being influenced by the loading effects of the body. The induced voltage measured by the small RF coil is used to control the phase and amplitude of the RF power supplied to the resonant coil. The use of such small RF coils is particularly useful in cases in which the resonant RF coil comprises multiple coil elements, which are driven e.g. like a phased array. In this case it is possible to control the amplitude and phase of the RF power supplied to each of the individual coil elements in a way that they realize a very uniform RF field inside the body to be imaged, when they are driven simultaneously.

### SUMMARY OF THE INVENTION

It is an object of the present invention to provide a high quality MRI technique. In particular it is an object of the present invention to provide a MRI technique, in which the influence of physiological factors such as respiration and cardiac pulsation on MRI results is reduced or removed. The object of the present invention is achieved by a magnetic resonance imaging system, comprising a first RF coil adapted for acquiring magnetic resonance imaging data of a patient's body; a number of measuring elements, which are sensitive to a load changing of the first RF coil, said measuring elements being adapted for acquiring data related to motion of said patient's body; and a processing unit adapted for employing said motion data to correct for patient motion in magnetic resonance imaging.

The loading of the resonance coil(s) are affected by movements of the body to be imaged. Unintended movements of the body are for example movements of the patient's chest due to respiration or movements due to cardiac pulsation.

This object is also achieved according to the invention by a magnetic resonance imaging method, comprising the steps of: acquiring magnetic resonance imaging data of a patient's body by means of a first RF coil; acquiring data related to motion of said patient's body by means of a number of measuring elements, which are sensitive to a load changing of the first RF coil; and employing said motion data to correct for patient motion in magnetic resonance imaging by means of a processing unit.

The object of the present invention is also achieved by a computer program for carrying out the above mentioned method, said program comprising computer instructions to employ said motion data to correct for patient motion in magnetic resonance imaging by means of a processing unit, when the computer program is executed in a computer. The technical effects necessary according to the invention can thus be realized on the basis of the instructions of the computer program in accordance with the invention. Such a computer program can be stored on a carrier such as a CD-ROM or DVD or it can be available over the internet or another computer network. Prior to executing the computer program is loaded into the computer by reading the computer program from the carrier, for example by means of a CD-ROM player or DVD player, or from the internet, and storing it in the memory of the computer. The computer includes inter alia a central processor unit (CPU), a bus system, memory means, e.g. RAM or ROM etc., storage means, e.g. floppy disk or hard disk units etc. and input/output units. Alternatively, the inventive method could be implemented in hardware, e. g. using one or more integrated circuits.

A core idea of the invention is to provide a technique for reducing or removing the influence of physiological factors such as respiration-related abdominal motion or cardiac motion due to cardiac pulse or both on MRI results. As a result, the invention allows e.g. to compensate for image-to-image fluctuation due to physiological motion of an object being imaged. For this purpose the respiratory phase and/or the cardiac phase of the patient is determined by detecting patient motion during the MR imaging process. Motion detection is carried out by determining the changing loading effects on the first RF coil(s) (or on coil elements thereof) by measuring the changes in voltages that are induced in measuring elements, which are sensitive to a load changing of the first RF coil.

Artifacts due to physiological motion in anatomic imaging are well recognized, and an assortment of techniques have been developed for their reduction. The most straightforward approach is to synchronize the data acquisition to the specific motion by gating or triggering. According to the present invention, the knowledge of the respiratory phase and/or the cardiac phase of the patient, at the time of the RF pulse, is subsequently used to modify acquisition properties of the imaging method (i.e. encoding order, field of view, slice position, flip angle of next pulse, etc.) on-the-fly, i.e. during MRI data acquisition. In other words, the MRI data and the data related to patient motion are acquired simultaneously with RF transmission, and the MRI sequence is adapted immediately after the RF transmission.

According to another aspect of the invention, the knowledge of the respiratory and/or the cardiac phase of the patient during imaging allows retrospective synchronization of imaging data with physiological activity during data processing, i.e. if MRI data acquisition is completed. In this case imaging data are retrospectively ordered into physiological cycles (e.g. respiratory and cardiac cycles). Afterwards the physiological effects are removed from the MRI data. In other words, according to this aspect of the invention, physiological activities of the subject are monitored while imaging data are being acquired, and then retrospectively the physiological effects are estimated and removed as guided by the acquired physiological data.

A main advantage of the proposed invention compared to existing methods to consider physiological activity (i.e. mechanical bellows strapped around the patients chest) is that it requires no additional apparatus to be placed on the patient. In addition, as the patient is moved through the system, motions other than breathing (or cardiac motion) can be detected. For example, if the patient moves suddenly during a scan, this will be detected and the MR data acquisition can be compensated accordingly. Furthermore, the present invention is insensitive to changes in the duration of each physiological cycle (respiration cycle, cardiac cycle, etc.) and can be used under various experimental conditions.

These and other aspects of the invention will be further elaborated on the basis of the following embodiments which are defined in the dependent claims.

According to a preferred embodiment of the invention the measuring elements are adapted for acquiring respiration motion and/or cardiac motion of the patient. For this purpose a RF pickup coil is used as measuring element, which is sensitive to loading effects to the first RF coil or it's elements. A good sensitivity to loading effects can be achieved, if the RF pickup coil is positioned in close proximity to the first RF coil. If such a RF pickup coil is employed, the main sources of motion errors during MRI measurements are captured. In other preferred embodiments of the invention, instead of (or in addition to) the RF pickup coil, a directional coupler and/or an electric or electronic component having predetermined electromagnetic properties may be used as measuring element.

According to a preferred embodiment of the invention the first RF coil is a multi-element RF coil. With such an RF coil a homogeneous RF field can be obtained inside a patient's body even at higher frequencies and an increased RF strength. If each RF coil element comprises its own measuring element, the motion detection can be carried out in a very accurate way. Alternatively, the number of measuring elements is not equal to the number of RF coil elements. Preferably the number of measuring elements is smaller than the number of RF coil elements. This may be desirable when the patient motion to be measured is characterized by a few degrees of freedom. In this case, a smaller number of measuring elements provides a reduction on the complexity and cost of the system.

According to another preferred embodiment of the invention the multi-element RF coil is adapted to be positioned directly on the patient and is removable from the MRI system. In other words, not only a fixed (system integrated) volume RF coil, e.g. in form of a transmit array, may be used. Instead, a local (preferably removable) transmit coil topology, e.g. local transmit coil arrays, might be used as well.

The present invention can also be used if the first RF coil is a quadrature birdcage coil and the measuring elements are arranged to detect changes in loading of the horizontal and vertical (orthogonal) resonant modes of the first RF coil. This approach can be applied, as a retrofit, on existing MRI systems.

### BRIEF DESCRIPTION OF THE DRAWINGS

These and other aspects of the invention will be described in detail hereinafter, by way of example, with reference to the following embodiments and the accompanying drawings; in which:

FIG. 1 shows a schematic illustration of a MRI system,

FIG. 2 shows a diagram schematically illustrating different steps of the inventive method on a time scale,

FIG. 3 shows a schematic illustration of a multi-element transmit RF coil system including pickup coils,

FIG. 4 shows a schematic structure of a single channel of the RF coil system with a pickup coil,

FIG. 5 shows a schematic structure of a single channel of the RF coil system with a direction coupled,

FIG. 6 shows a schematic structure of a single channel of the RF coil system with direct measurement using a capacitor.

### DETAILED DESCRIPTION OF THE EMBODIMENTS

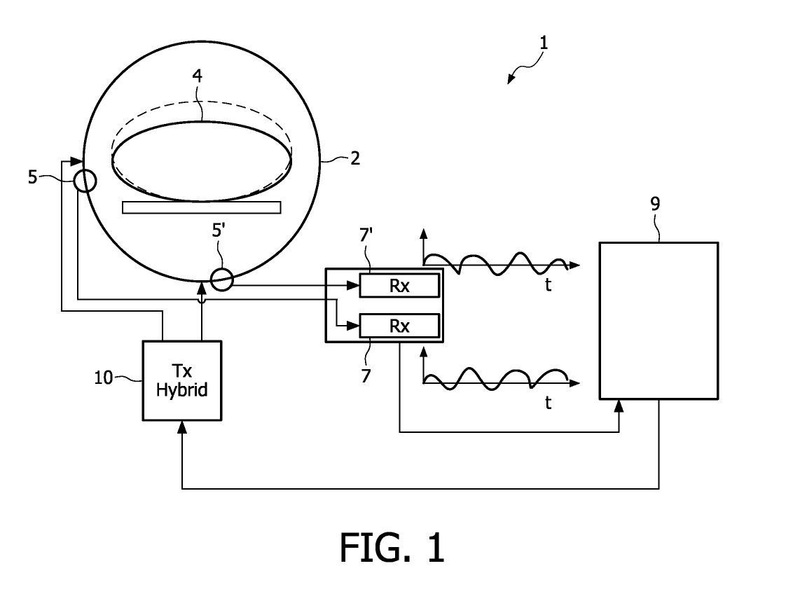

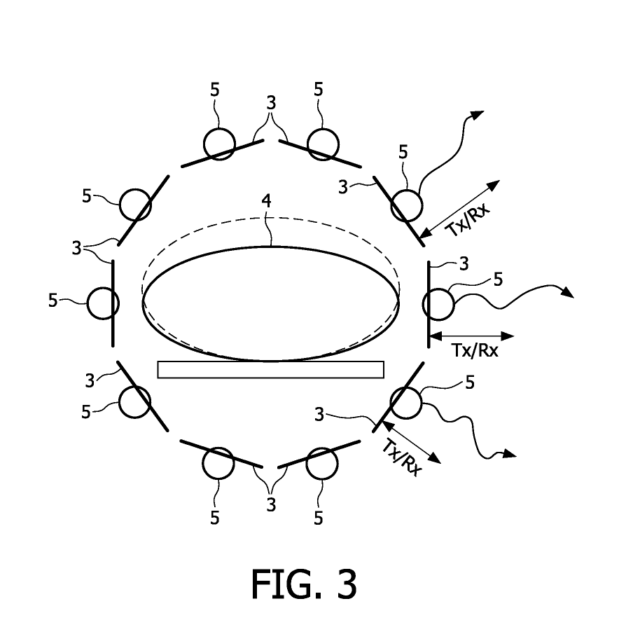

A simple embodiment of the present invention is described below. A MRI system 1 comprises a volume multi-element transmit/receive (or even transmit only) RF coil 2 with multiple RF coil elements 3 (not shown in FIG. 1; see FIG. 3). In particular the MRI system 1 comprises at least two RF coil elements 3 adapted for acquiring magnetic resonance imaging data of a patient's body 4. Each RF coil element 3 is designed to incorporate an independent second RF coil (pickup coil) 5, which serves as measuring element.

Each pickup coil 5 is physically adjacent to one of the at least two RF coil elements 3, and is adapted for acquiring data related to motion of said patient's body 4. The number of pickup coils 5 form a pickup coil array. Each pickup coil 5 is connected to a receiver 5 (detector electronics) for determination of the actual (or relative) magnetic field produced by each RF coil element 3 under different loading conditions. The measuring of voltages by the pickup coils 5 will be carried out at RF frequencies. The number of receivers 7 form a receiver array. The number of receivers 7 are connected to a processing unit 9. The processing unit 9 is adapted for employing said motion data to correct for patient motion in magnetic resonance imaging, as described in more detail below.

FIG. 1 illustrates a schematic diagram of the inventive system using a generic transmit RF coil 2. Furthermore, a patient 4 in two different breathing states is illustrated. According to the invention a number of pickup coils 5 are positioned next to the transmit coil elements 3 (not shown in FIG. 1). Here the transmit RF coil 2 contains a first pickup coil 5 and a second pickup coil 5′. Each pickup coil 5, 5′ is connected to a receiver 7 from which it is possible to extract the representative voltage amplitude (and phase), in real-time, for each pickup coil 5, 5′.

In prior art use, these pickup coils 5, 5′ feedback local RF amplitude (and phase) data that can be used to provide calibration information for correct adjustment of the RF field amplitude and phase for each RF coil element 3. In addition, the pickup coils 5, 5′ can be used to provide a safety mechanism against over exposure of the patient 4 to RF by one or more transmit RF coil elements 3.

The present invention relates to an additional use of the information that can be obtained from the array of pickup coils 5, 5′. Each RF pulse that is transmitted by the multi-element RF transmit coil 2 leads to an induced voltage in each pickup coil 5, 5′. The voltage amplitude, and phase, induced in any specific pickup coil 5, 5′ will be dominated by the RF field generated by the closest transmit RF coil element 3. For a fixed power to each transmitting RF coil element 3, the induced voltage amplitude in each pickup coil 5, 5′ will also depend upon the local loading conditions of each RF coil element 3. Since each pickup coil 5, 5′ is located next to and therefore associated with a particular RF coil element 3, from the measuring values (voltage) obtained from those pickup coils 5, 5′ it can be seen how well that particular RF coil element 3 is loaded by the patient's body. The proximity of the patient 4 to the RF coil element 3 will modulate the voltage on the pickup coil 5, 5′ in a way that allows to determine, whether the patient 4 is closer or further away from the RF coil element 3. If the patient breaths in, the chest expands and approaches the RF coil elements 3. For each RF coil element 3, which is approached by the patient's chest, the voltage of the associated pickup coil 5, 5′ will change, and will be modulated according to the breathing pattern of the patient 4. As a result, using the voltages measured by the pickup coils 5, 5′, the breathing motion and subsequently the breathing cycle of the patient 4 can be detected.

During a representative MRI sequence execution, an RF pulse is transmitted via the transmit RF coil 2. A controller 10 is used for controlling the transmit RF coil elements 3 of the MRI system 1. The RF pulse induces a voltage in each pickup coil 5, 5′ according to the loaded properties of the RF coil elements 3. The signals measured by means of the pickup coils 5, 5′ are not NMR signals, but are directly induced voltages due to the current flowing in the RF coil element, which depend on the loading of the RF coil 2. As the patient breathes out (dotted line) the voltage amplitude on the second pickup coil 5′ (detected during the RF pulse transmission) will increase since the body is moving further away from the RF coil elements in the vertical direction. The voltage amplitude on the first pickup coil 5 may also change as the vertical cross-section changes. It is likely, however, that the voltage on the second pickup coil 5′ exhibits the largest change. Since breathing is periodic, the voltage waveform may be sinusoidal in nature.

In other words, when a patient 4 is present inside the rigid multi-element volume transmitting RF coil 2, the respiratory motion of the patient 4 causes the position of various body parts to periodically move towards and away from various RF coil elements 3. This motion results in a change in the local loading conditions of each RF coil element 3. The difference in loading, per RF coil element 3, as a function of patient respiratory (and possibly other) motion, is reflected in the induced voltage amplitude in each pickup coil 5, 5′ during the application of an RF pulse.

The spatial distribution of voltage amplitudes over all the pickup coils 5, 5′, during RF transmission, is used to determine the respiratory phase of the patient 4 in real-time. For this purpose, following the RF excitation the sampled pickup coil signals are processed in the processing unit 9 to extract information pertaining to the position of, for example, the chest of the patient 4 during the RF pulse, i.e. the voltage in the second pickup coil 5′ will be low if the chest is expanded and high if contracted. The motion information is provided to the processing unit 9 either prior to or during data acquisition, for the purpose of adapting various properties of the MR pulse sequence on-the-fly. According to the measured voltage, it is possible to choose, for example, a specific encoding step for the subsequent MR data acquisition step that minimizes the effects of motion according to a preferable scheme relating k-space sampling to chest wall position. Additionally, the relative change in FOV can be estimated from the change in pickup coil voltage such that the measurement gradient amplitude can be changed, on-the-fly, to compensate. These steps will be explained in more detail below.

The same basic principles apply in case of observing the cardiac motion or any other motion of the patient 4 instead of or in addition to the respiratory motion. It should also be clear that when the information from breathing motion is available, all current MRI methods that utilize the information from respiratory motion (i.e. via mechanical bellows) equally apply.

The most convenient and preferred topology is to connect each pickup coil 5, 5′ to an individual receiver 7, 7′ such that the received signal is demodulated and available under full control of the data processing software executed in the processing unit 9. In another embodiment (not shown), the pickup coil voltage can be rectified using a diode circuit and fed into a comparator for reporting the voltage levels via a standard interface to the processing unit 9.

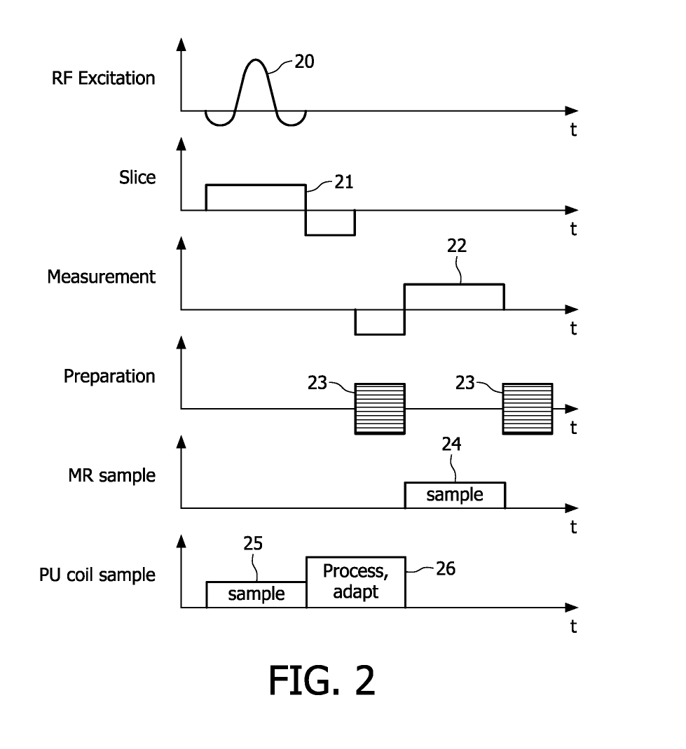

With respect to FIG. 2 different steps of the inventive method will now be explained. First, an RF pulse (RF waveform 20) is transmitted via the transmit RF coil. The RF waveform 20 is shown on the “RF Excitation” chart. The shape of a gradient waveform being transmitted in this case, i.e. the gradient sequence 21 of the MRI system, is shown in the “Slice” chart. In the “Measurement” chart the waveform of the measurement gradient 22 is shown, which is used for measuring the MR signal after excitation of the magnetic resonance. The preparation gradients 23 (encoding gradients) are shown in the “Preparation” chart. The RF waveform 20 illustrated in the “RF excitation” chart and the gradient sequence 21 are used to select the slice in the patient 4. The “Measurement” and the “Preparation” charts are used for reading out the MR signal.

In the “MR sample” chart the sampling of NMR data is illustrated, i.e. receiving RF energy from the RF coil. A “sample” region 24 is shown, where MR data are sampled during a measurement. This sampling is repeated a number of times.

In the “pickup coil sample” chart below it is illustrated, that the sampling of the voltage on the pickup coils (block “sample” 25) is performed during the RF excitation. Before the time of sampling MR data, a “process adapt” block 26 is shown in the “pickup coil sample” chart, which indicates, that between the RF sampling and the MR signal sampling, there is time to adapt the MR sampling scheme. In other words, during this time, it is possible to process MR data with the sample data and to extract any motion information. For example, it is determined from the sampled RF signal, where the position of the patient 4 is in e.g. the breathing cycle. Based upon that information a decision is made by means of the processing unit 9, e.g. using a lookup table or the like. As a result, a certain measurement waveform shape 22, or a certain preparation gradient shape 23 is selected. The steps performed during the “Process adapt” block 26 are carried out by means of the processing unit 9, which is connected to the RF transmit coil elements 3 via controller 10 to form a closed control circuit.

Subsequently, the processing results can either be stored by means of the processing unit 9 and a data storage (not shown) for later image reconstruction; or the processing results can be used to modify the two gradient channels, i.e. to modify the current image acquisition, by means of the processing unit 9. In the later case, the measurement gradient 22 and the preparation gradient 23 are changed according to the results of the sampling, which has been carried out during the RF excitation. If for example the processing of the pickup coil measurements reveals that the patient 4 is currently breathing out, a particular encoding status of the preparation gradient 23 can be selected at this point in time.

FIG. 3 shows a schematic illustration of a multi-element transmit/receive (Tx/Rx) RF coil 2 with ten RF coil elements 3. The patient 4 is surrounded by ten independent RF coil elements 3, each including a separate pickup coil 5 positioned adjacent to the RF coil elements 3.

Although in the embodiments illustrated above, at least two pickup coils 5, 5′ have been employed, the present invention may also operate with only one pickup coil 5. In that case, some assumptions have to be made about the physical motion of the patient 4, so that the pickup coil 5 can positioned in a place which is most sensitive to detecting a particular kind of motion.

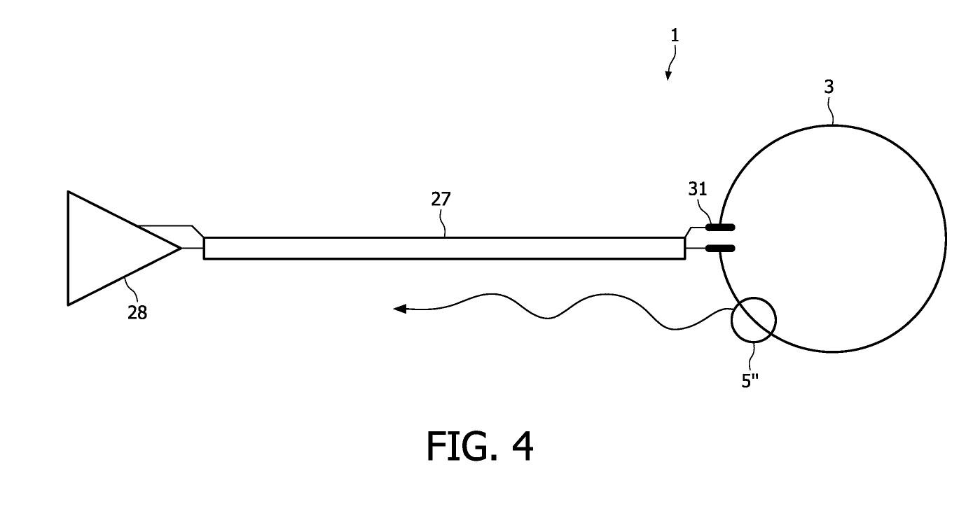

FIG. 4 shows the schematic structure of a single channel of multi-channel RF transmit system 1. For each channel the system uses a single pickup coil 5″ connected by coaxial cable 27 to a RF amplifier 28, which is part of the transmit chain. In other words, an independent RF amplifier 28 is used for each RF coil element 3. The transmitter and other parts of the system 1 are not shown in FIG. 4. In this example, the pickup coil 5″ is placed in close proximity to the RF coil 2 so that the alternating current in the conductor of the RF coil 2 induces a voltage in the pickup coil 5″ which can be monitored. As the RF coil 2 is loaded by a patient, the current in the RF coil 2 is modulated and the pickup coil 5″ senses this via the inductive coupling and corresponding change in voltage. In other words, when the RF amplifier 28 transmits, the forward and reflected power is used to indicate the state of the load.

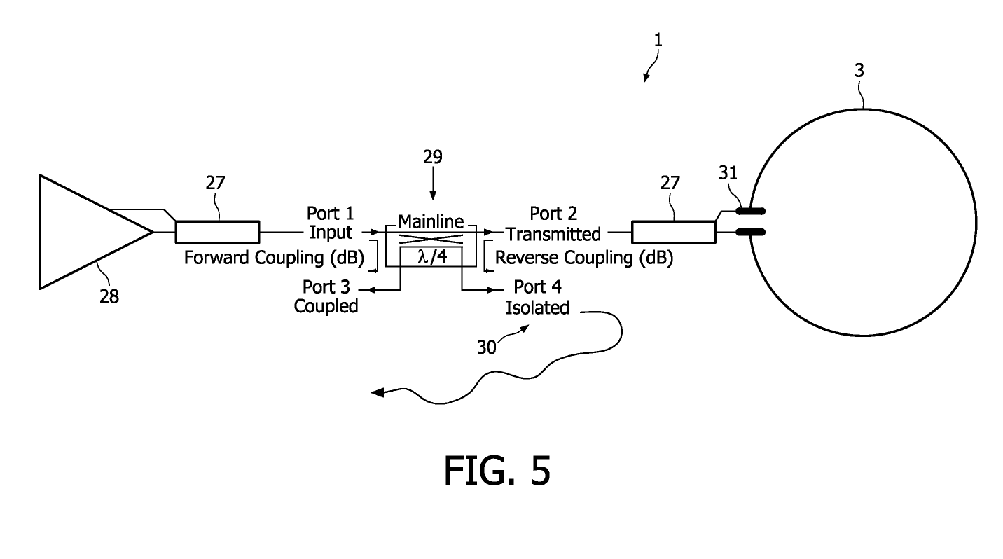

FIG. 5 shows an alternative embodiment of the invention in which the pickup coil in each channel of the system is replaced with a directional coupler 29. The proportion of RF power transmitted to the load is referred to as the “forward” power. The proportion of RF (electrical) power reflected from the load is referred to as the “reflected” power. The directional coupler 29 now senses a portion of the forward and reflected power between the RF amplifier 28 and the RF coil element 3. When the load of the RF coil element 3 changes, the impedance of the RF coil 2 changes and this results in a change in the measured reflected power. Thus, indirectly, the changing load on the RF coil element 3 can be monitored by measuring the reflected power voltage from the directional coupler 29, in particular by measuring the voltage on the reflected power port 30.

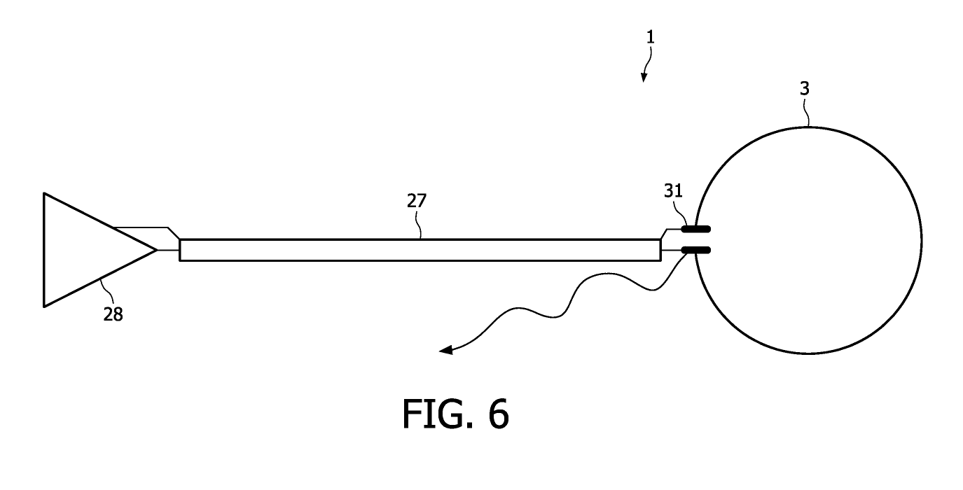

The RF coil element 3 is a resonant structure which uses a copper loop (inductor) often in series with a capacitor 31. Together they resonate and energy is exchanged between inductor and capacitor. Using a pickup coil 5 close to the RF coil 2 the changing current in the copper loop of the RF coil (inductor) can be monitored via inductive coupling, which is measured as a voltage change on the pickup loop, see above. This same voltage modulation can, however, be observed by directly measuring the voltage across the capacitor 31. Thus, FIG. 6 shows an alternative embodiment of the invention in which for each channel of the system the voltage across a component having predetermined electromagnetic properties such as capacitance, inductivity, resistance etc. (e.g. a fixed capacitor 31) is measured directly. This voltage is proportional to the loading of the RF coil 2. In this case, however, the voltage may be higher and it is necessary to be careful not to add resistance that can spoil the performance of the RF coil resonance.

All appliances described are adapted to carry out the method according to the present invention. All devices, in particular the processing unit 9, are constructed and programmed in a way that the procedures for obtaining data and for data processing run in accordance with the method of the invention. The processing unit 9 is adapted for performing all tasks of calculating and computing the measured data as well as determining and assessing results. This is achieved according to the invention by means of a computer software comprising computer instructions adapted for carrying out the steps of the inventive method, when the software is executed in the processing unit 9. The processing unit 9 itself may comprise functional modules or units, which are implemented in form of hardware, software or in form of a combination of both. In a preferred embodiment of the invention the processing unit 9 is realized in form of a microcomputer.

It will be evident to those skilled in the art that the invention is not limited to the details of the foregoing illustrative embodiments, and that the present invention may be embodied in other specific forms without departing from the spirit or essential attributes thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein. It will furthermore be evident that the word “comprising” does not exclude other elements or steps, that the words “a” or “an” do not exclude a plurality, and that a single element, such as a computer system or another unit may fulfil the functions of several means recited in the claims. Any reference signs in the claims shall not be construed as limiting the claim concerned.

REFERENCE NUMERALS

| | |
|---|---|
| 1. | MRI system |
| 2. | RF coil |
| 3. | coil element |
| 4. | patient's body |
| 5. | pickup coil |
| 6. | (free) |
| 7. | receiver |
| 8. | (free) |
| 9. | processing unit |
| 10. | controller |
| 20. | RF pulse |
| 21. | gradient sequence |
| 22. | measurement gradient |
| 23. | preparation gradient |
| 24. | MR sample region |
| 25. | pickup coil sample region |
| 26. | process adapt region |
| 27. | coaxial cable |
| 28. | RF amplifier |
| 29. | directional coupler |
| 30. | reflected power port |
| 31. | capacitor |

1. A magnetic resonance imaging system, comprising a first RF coil adapted for acquiring magnetic resonance imaging data of a patient's body;
a number of measuring elements, which are sensitive to a load changing of the first RF coil, said measuring elements being adapted for acquiring data related to motion of said patient's body; and
a processing unit adapted for employing said motion data to correct for patient motion in magnetic resonance imaging.

2. The magnetic resonance imaging system as claimed in claim 1, wherein the load sensitive measuring elements are adapted for acquiring respiration and/or cardiac motion of the patient.

3. The magnetic resonance imaging system as claimed in claim 1, wherein the first RF coil is a transmit only coil or a transmit/receive coil.

4. The magnetic resonance imaging system as claimed in claim 1, wherein the first RF coil is a multi-element RF coil.

5. The magnetic resonance imaging system as claimed in claim 4, wherein each RF coil element comprises its own load sensitive measuring element.

6. The magnetic resonance imaging system as claimed in claim 4, wherein the number of load sensitive measuring elements is not equal to the number of RF coil elements.

7. The magnetic resonance imaging system as claimed in claim 4, wherein the multi-element RF coil is adapted to be positioned directly on the patient.

8. The magnetic resonance imaging system as claimed in claim 1, wherein the first RF coil is a quadrature birdcage coil and the load sensitive measuring elements are arranged to detect changes in loading of the horizontal and vertical resonant modes of the first RF coil.

9. The magnetic resonance imaging system as claimed in claim 1, wherein load sensitive measuring elements comprises second RF coils and/or directional couplers and/or an electric or electronic component, coupled to the RF coil.

10. A magnetic resonance imaging method, comprising the steps of:
acquiring magnetic resonance imaging data of a patient's body by means of a first RF coil;
acquiring data related to motion of said patient's body by means of a number of measuring elements, which are sensitive to a load changing of the first RF coil; and employing said motion data to correct for patient motion in magnetic resonance imaging by means of a processing unit.

11. The magnetic resonance imaging method as claimed in claim 10, in which the magnetic resonance imaging data and the data related to patient motion are acquired simultaneously with RF transmission.

12. The magnetic resonance imaging method as claimed in claim 10, in which the correcting step comprises adapting the MRI sequence after the RF transmission.

13. A computer program for carrying out a magnetic resonance imaging method; in which method magnetic resonance imaging data of a patient's body being acquired by means of a first RF coil; and in which method data related to motion of said patient's body being acquired by means of a number of measuring elements, which are sensitive to a changing of the load of the first RF coil; the program comprising computer instructions to employ said motion data to correct for patient motion in magnetic resonance imaging by means of a processing unit, when the computer program is executed in a computer.
